(19) United States
(12) Patent Application Publication
(10) Pub. No.: US 2026/0057981 A1
(43) Pub. Date: Feb. 26, 2026

(54) ASSISTIVE PROBLEM-ORIENTED SUMMARY GENERATION OF PATIENT RECORDS

(71) Applicant: Oracle International Corporation, Redwood Shores, CA (US)

(72) Inventor: Harshit K. Kalley, Santa Clara, CA (US)

(73) Assignee: Oracle International Corporation, Redwood Shores, CA (US)

(21) Appl. No.: 18/811,404

(22) Filed: Aug. 21, 2024

(51) Int. Cl. G16H 15/00; G06F 16/34; G16H 10/60

(52) U.S. Cl. CPC G16H 15/00; G06F 16/345; G16H 10/60

(57) ABSTRACT

A communication that is associated with a subject is accessed via an interface. One or more clinical concepts are determined based on the communication by using natural language processing and an ontological knowledge graph, where the ontological knowledge graph defines semantics, constraints, and relationships between a plurality of terms. An electronic health record is queried using the one or more clinical concepts. In response to the query, a set of records corresponding to the one or more clinical concepts from the electronic health record is received. An output is generated based on the set of records by using a generative artificial intelligence (GenAI) model, where the output comprises a summary of the set of records, a trend, or a categorization of information of the set of records. The interface is updated with the output.

### BACKGROUND

In healthcare environments, clinicians are frequently tasked with composing comprehensive summaries of patient health records for various reasons which may include administrative, legal, or procedural demands etc. For instance, after consultation with a physician, a patient may be provided with an after-visit summary. Similarly, for hospitalized patients, daily progress reports may be generated for accurate medical management. Additionally, an anticipatory or a pre-visit summary that may cover prior health records of the patient can be generated to enhance the efficiency of physician-patient encounters. Furthermore, comprehensive overviews of treatments administered by other specialists can be beneficial for general practitioners and specialists alike and may facilitate informed decision-making and continuity of care. However, manual compilation of such summaries may cause a substantial administrative toll and can be time consuming for the clinicians.

Moreover, the retrieval of pertinent information from disparate sources via diverse interfaces may pose additional challenges. For instance, in the case of virtual consultations (e.g., via secure messaging platforms) a clinician may receive a message with limited context via an email from a patient. In order to make informed decisions, for example generating prescriptions based on the message with limited context, the clinician may navigate various other interfaces to access patient histories and review past clinical data. Subsequently, the clinician may communicate back the informed decisions typically through email or other secure messaging platforms.

Therefore, there is a need for a system, methods, or techniques that can understand medical terminologies, automatically retrieve relevant or problem-specific data, generate concise summaries or collate relevant patient information preferably within interfaces of the secure messaging platform. More specifically, an automated system or tool that can generate problem-oriented (e.g., a specific disease or a health problem) summaries, trends, and classification of information in categories as per clinician preferences may facilitate prompt and well-informed replies by the clinicians. Such an automated system can have the potential to mitigate administrative burdens, reduce time expenditures, and may enhance both the efficiency of healthcare delivery and the quality of patient care.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 shows an exemplary network 100 for performing a method to generate assistive problem-oriented summary based on patient records in accordance with some embodiments of the present disclosure.

FIG. 2 shows an exemplary block diagram of a system to generate a problem-oriented summary, a trend, or a categorized information based on a set of records of the subject and by using a GenAI model.

FIG. 3A shows an illustrative example of a first interface of a user interface of an application that can be run on a user device in accordance with some embodiments of the present disclosure.

FIG. 3B shows an illustrative example of the second interface of the user interface of the application that can be run on the user device

FIG. 4 shows an example flowchart of a system to generate the problem-oriented summary, the trend, or the categorized information based on the set of records of the subject in accordance with some embodiments of the present disclosure.

FIG. 5 illustrates a simplified diagram of an example distributed system for a cloud hosting a GenAI platform.

FIG. 6 is a simplified block diagram of a cloud-based system environment in which various services of a server of FIG. 5 may be offered as cloud services, in accordance with some aspects of the present disclosure.

FIG. 7 illustrates an exemplary computer system to implement some aspects of the present disclosure.

### DETAILED DESCRIPTION

Some embodiments of the present disclosure relate to generation of a summary using generative artificial intelligence (GenAI) cloud platform and based on a set of records of a subject (also referred herein as patient) that are selected using various selection criteria. The various selection criteria may include but are not limited to author of a record, time interval or duration, record type (e.g., lab reports, inpatient or outpatient clinical notes etc.), and visit or encounter type (e.g., specific disease or problem, specific department such as cardiology, urology etc.). In some instances, the various selection criteria may further include symptoms, problems, or clinical conditions that are automatically extracted from a message, communication, or an email of the subject that may be sent for virtual consultation. Moreover, the generated summary can be reviewed or customized by a clinician before approval and/or signing (e.g., digital signatures) by the clinician.

According to some embodiments, healthcare professionals (e.g., clinicians, physicians, or nurses) may record health information of a subject during various encounters. Each record can be stored in a database with an identifier and metadata. The metadata may include information about the subject, author of the record, encounter type, record type etc. The database can be an electronic health record (EHR) database, an electronic medical record (EMR) database, a cloud-based database, a local database of an organization and the like. A user or a clinician may select criteria using a clinician interface or a user interface. Based on the selected criteria, the set of records or documents may be retrieved from the database or the EHR. In some instances, a prompt may be generated automatically for a GenAI model based on the selected criteria, the set of records, and/or user preferences. The user preferences may include choices related to output of the GenAI model such as output type (e.g., summary, trend), or style preferences, for example, language or formatting style, or purpose of the summary (discharge, insurance claim, case referral etc.).

An inference request that may include the prompt and the set of records may be sent to the GenAI cloud platform. In some cases, the inference request may include the prompt and the identifier for each record of the set of records. The GenAI cloud platform may provide access to one or more GenAI models through a GenAI service. The one or more GenAI models may include but not limited to large language models (LLMs) such as Llama, GPT, Gemini, Grok etc. The one or more GenAI models may further include retrieval-augmented generation (RAG) model, a fine-tuned transformer model, or a domain-specific model that is trained on domain-specific data. A summary or problem-oriented summary of the set of records may be generated by executing the inference request using the GenAI service and a GenAI model of the one or more GenAI models. The summary can be displayed on the user interface to review, edit, and/or to sign by the user.

In some embodiments, one or more clinical concepts and/or context may be extracted automatically from a message, communication, or an email of the subject. For example, the subject may send an email to the user or clinician for virtual consultation. For instance, a snippet of the email can be ‘. . . Overall, I am feeling better after using the prescribed medication on my last visit, however I started getting blood in urine since yesterday . . . ’. The text in the email may be analyzed using natural language processing (NLP) to extract the context or at least one clinical condition, for example, last visit of the subject, blood in urine etc. The extracted context and/or the clinical condition may be processed further using the NLP and an ontological knowledge graph to determine one or more clinical concepts such as associated symptoms, problems, medications, related diseases and the like. In case of above example, the one or more clinical concepts that can be extracted and not limited to are: ‘ICD10 R31.9 Hematuria, unspecified’, under problems category, or ‘SNOMED 34436003 blood in urine’ under symptoms category.

The term ‘ontological knowledge graph’ as used herein, may refer to structured representations of knowledge that may use domain-specific ontologies or clinical ontologies to define entities (e.g., diseases, symptoms, medications), relationships (e.g., causes, treatments), and constraints (e.g., dosage limits, contraindications). The ontological knowledge graph may define semantics, constraints, and relationships between a plurality of terms. The plurality of terms can be clinical terms and non-clinical terms. The relationship generally includes at least a clinical term in each relationship. For instance, diabetes (a clinical term) may be represented by a node that can be connected with the nodes representing medications to treat diabetes in the ontological knowledge graph. Each term, clinical or non-clinical, may be connected or associated with any number of other terms. The ontological knowledge graph may also include lexical variants of the terms. Exemplary lexical variants may include ‘belly pain’ (a non-clinical term for ‘abdominal pain’ 21522001 SNOMED-CT), ‘belly ache’ (for ‘abdominal pain’ 21522001 SNOMED-CT), or ‘belly ache’ (for ‘abdominal discomfort’ 43364001 SNOMED-CT) and the like. The ontological knowledge graph may utilize existing standard terminologies such as systematized nomenclature of medicine-clinical terms (SNOMED-CT), logical observation identifiers names and codes (LOINC), or international classification of diseases, 10^{th} revision (ICD10) for interoperability.

According to some embodiments, the clinical condition, the context, and/or the one or more clinical concepts may be used to search relevant records in the EHR. The search can be performed as a simple keyword matching based search or using advanced techniques such as representing documents or records as vectors and computing similarity metrics with search terms. Afterwards, selecting the records that exhibit a higher degree of similarity than a predefined threshold. In some instances, in addition to above mentioned search terms (e.g., the clinical condition, the context, and/or the one or more clinical concepts) the selection criteria (e.g., last 3 months, lab tests only), as selected by the user, may be utilized to search relevant records. Afterwards, the search results, for example, the set of records and/or the search criteria may be utilized to generate the inference request including the prompt. The inference request can be processed by using the GenAI model. The GenAI model may leverage the set of records or retrieved information to generate an augmented context or detailed context by combining the retrieved information with the prompt. The augmented context based on the set of records may provide additional information or knowledge to the GenAI model as compared to training data on which the GenAI model was pretrained. The GenAI model (e.g., RAG model) may generate responses or the output that is based on the retrieved (factual) information (i.e., the set of records). In some instances, relevant data or the set of records may be retrieved by the GenAI model.

According to some aspects of the present disclosure, the output of the GenAI model may be comprised of the problem-oriented summary, categorized information, a trend, or a prescription suggestion. In some instances, the GenAI model may be used to categorize information into different sections such as symptoms, vitals, previous diseases, prescriptions, and the like. In some instances, a score may be assigned to each record of the set of records based on a degree of relevancy with the one or more clinical concepts and/or the selected criteria. Moreover, recent documents or records may be assigned relatively higher score as compared to old records. The score can be used to resolve conflicts (if any) in the information of the set of records by giving priority to information from a high score record. During categorization of information, the GenAI model may remove duplication of terms or may highlight (e.g., making the text bold) for any contradictory terms, or test results. Moreover, the output may include the trend to depict temporal variation in symptoms, biomarkers (or entities in test reports), prescribed medications and the like. In some other instances, the GenAI model may provide a prescription recommendation based on prescriptions data or records from other physicians for same symptoms or disease. For example, according to temporal analysis, if the symptoms are not improving with the current or recently prescribed medications in last few encounters, the GenAI model may generate recommendations for prescriptions, more lab tests, or even risk of potential other diseases.

The disclosed techniques can be implemented in the form of a backend service and a frontend widget and can be integrated in existing software or tools such as PowerChart™, MessageCenter™ and the like. The frontend widget may include sections in the user interface for the generated summary, trends, categorized information, or the various selection criteria. The user may select various filters or criteria using the frontend widget. The backend service may query and filter the records or documents from the EHR database. The backend service may utilize role-based access control (RBAC) and identity verification systems to authenticate and authorize user requests or clinician requests. Moreover, the backend service can be further extended by pairing with other platform services or cloud services such as oracle cloud infrastructure (OCI), amazon web service (AWS) and the like. The backend service may be further comprised of a representational state transfer (REST) based service that can accept documents, extract content and may call a GenAI service application programming interface (API) of the cloud platform with appropriate credentials. The REST service can also implement functions to measure the number of tokens and can chunk the content into right size batches in order to meet limitations and/or rate limits of the GenAI service API.

In some instances, upon selection or retrieval of documents or records, a draft summary can be generated by clicking the generate button from the frontend widget. Original text of the documents and the generated draft summary can be shown in side-by-side windows or pop-up windows. The frontend widget may include features or buttons for the user to verify, sign, and save the summary as a record. The user workflow can emphasize human intervention (e.g., the clinician) to enforce patient safety.

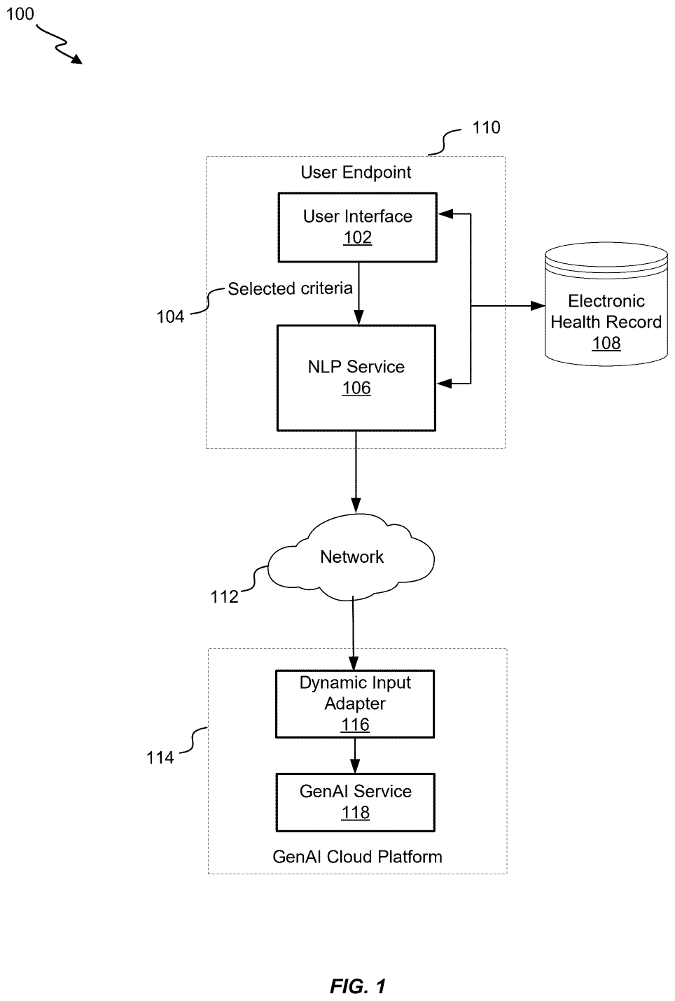

FIG. 1 shows an exemplary network 100 for performing a method to generate assistive problem-oriented summary based on patient records in accordance with some embodiments of the present disclosure. Exemplary network 100 comprises a user endpoint 110, a network 112, a GenAI cloud platform 114, and an electronic health record (EHR) 108 or a database. The user endpoint 110 may include a tablet, a laptop, a desktop computer, a computer server, and the like. The user endpoint 110 may run an application, a web-based application, or a cloud-app and may provide a user interface 102.

A clinician (also referred herein as user) may interact with the user interface 102 of the application to review, generate, store, and assess records of the subject. The user may further utilize the user interface 102 to read and respond messages from the subject registered on the application and hence provide communication capability within a given organization. The user may store recently generated records (e.g., documents or notes) and/or pull the records from the database or the EHR 108. The user interface 102 may also include components to receive information corresponding to selected records of the subject or one or more subjects from another user or a caregiver. Examples may include historical data, laboratory reports, past and current medications and prescriptions, encounter or visit reasons, and the like. The user interface 102 may represent an interface of an application that may be authorized and registered to use within a specific territory or may have limited access to other registered individuals. The user interface 102 can be a web-based application. In some instances, the user interface 102 may be a dedicated application with a custom designed graphical user interface (GUI), for example, PowerChart™ or MessageCenter™ application.

To generate an accurate problem-oriented summary, the user may select or choose criteria from the various selection criteria available on the user interface 102. Selected criteria 104 may be sent to an NLP service 106 for further processing. In some instances, in addition to the selected criteria 104, the message or communication received from the subject may also be sent to the NLP service 106. The message or communication received from the subject may describe recent or past encounters, symptoms, medications and the like. The selected criteria 104 may be comprised of filters such as time interval or duration based (e.g., last 24 hours or last four months), author of the record (e.g., clinician-wise), encounter type (e.g., nature of visit), or record type (e.g., lab test, prescriptions). For instance, in case of an ear, nose, and throat (ENT) specialist, the user (or the ENT specialist) may need to access encounter notes or laboratory reports related to ear problem only. In some instances, the user may decide not to provide the selected criteria 104 and only the message sent by the subject may be transmitted to the NLP service 106.

The NLP service 106 and the user interface 102 can be communicatively coupled to the EHR 108. The NLP service 106 may access the selected criteria 104 and/or the message (or communication, email etc.). The NLP service 106 may be employed to identify the context, intent, clinical conditions, or associated clinical concepts based on the message of the subject. Based on the identified context or concepts and the selected criteria 104, the NLP service 106 may retrieve the set of records of the subject from the EHR 108. The NLP service 106 may be comprised of a software application or set of applications, programs, routines, or computer performed services. The NLP service 106 may reside on the computing device of the user such as laptop, smartphone and the like. In some instances, the NLP service 106 may reside on a remote server communicatively coupled to the computing device of the user. In some other instances, the NLP service 106 may comprise an application or set of applications deployed in a cloud-based platform providing virtualized resources, for example, OCI, AWS, and Google cloud.

The EHR 108 can store a plurality of records of one or more subjects and may be comprised of one or more data storage devices across one or more computers and/or servers. The EHR 108 system may comprise one or a plurality of EHR systems such as hospital EHR systems, health information exchange EHR systems, clinical genetics/genomics systems, ambulatory clinic EHR systems, psychiatry or neurology EHR systems, insurance, collections or claims records and the like. In some instances, the EHR 108 may include one or more data stores of health-related records and may further include one or more computers or servers that facilitate the storing and retrieval of the records. In some instances, the EHR 108 or the database may be implemented as a cloud-based platform or may be distributed across multiple physical locations. The EHR 108 may further comprise of systems that can store real-time or near real-time information or data of the subject, for example, data from wearable sensors, bedside monitors, or in-home patient monitors or sensors. In some other instances, the EHR 108 may encompass multiple data storage units distributed across a network of interconnected computers and servers for better scalability, fault tolerance, and efficient data retrieval.

The user endpoint 110 may utilize the network 112 to communicate with the GenAI cloud platform 114. The network 112 may comprise of any form of communication network including public, private, internet, switch, routers, firewalls, and/or similar networks facilitating collaboration, information flow, and seamless connectivity between end nodes. In some embodiments, the network 112 may be a collection of interconnected devices, such as computers, servers, and routers, communicating with each other, enabling data exchange and resource sharing. In other embodiments of present disclosure, the network 112 may be a local area network (LAN) covering a small geographical area with high data transfer rates using ethernet cables or Wi-Fi. The network 112 may be a wide area network (WAN) covering extensive geographical distances and connecting multiple LANs together and/or may include a metropolitan area network (MAN) connecting multiple LANs within a specific organization territory such as hospitals, offices and the like. The other forms of the network 112 with reference to the present disclosure may include any campus area network (CAN), storage area network (SAN) and/or a virtual private network (VPN) to create a secure encrypted connection over a public network (usually the internet). Moreover, the selection of network 112 may depend on factors like scalability, security, and performance requirements.

The GenAI cloud platform 114 may provide the problem-oriented summary of the set of records through a GenAI service 118 and a dynamic input adapter 116. The dynamic input adapter 116 may process text inputs of varying lengths, such as sentences or documents (retrieved records in this case or the set of records), for functions or methods that work with individual tokens or sentences as arguments. During NLP-based document processing, tokenization may be performed at first by splitting the document text into tokens (e.g., words, phrases, or other meaningful units). In some instances, the dynamic input adapter 116 can be employed to unpack tokens from lists or arrays (e.g., received from the NLP service 106), making it easier to process each token individually or to pass them to the functions or methods. In other instances, the dynamic input adapter 116 can aid in unpacking feature vectors or transforming text features into a format suitable for the GenAI model. The dynamic input adapter 116 can also be utilized to simplify concatenation of text fragments or expansion of text representations into structured formats. The dynamic input adapter 116 may preprocess the set of records of variable length for flexible handling and then pass them to the GenAI service 118 for further actions such as generating summary, categorization of information, generating trends, and/or prescriptions suggestions.

The GenAI service 118 may provide access to one or more GenAI models that can generate content based on the patterns or information learned from the training data or data provided at run-time. In response to the prompts or input data (e.g., the set of records), the GenAI service 118 may utilize the one or more GenAI models to create the output such as the problem-oriented summary. In some embodiments of the present disclosure, the GenAI service 118 may include text-based GenAI services to output coherent paragraphs and summarize the provided contents. The one or more GenAI models may include but are not limited to generative pre-trained transformers (GPTs), Gemini, Grok, Llama and the like. The one or more GenAI models may be utilized to analyze data or records of the subject, for example, to generate a trend based on longitudinal data of the subject such as variations in symptoms over time, medications history, medications intake versus symptoms severity and the like. According to some embodiments of the present disclosure, executing the GenAI service 118 may refer to executing the GenAI model through cloud service providers, offering pre-built models, APIs and infrastructures. In other instances, the GenAI service 118 may include a custom GenAI model or solution that may be trained on propriety data or domain-specific data.

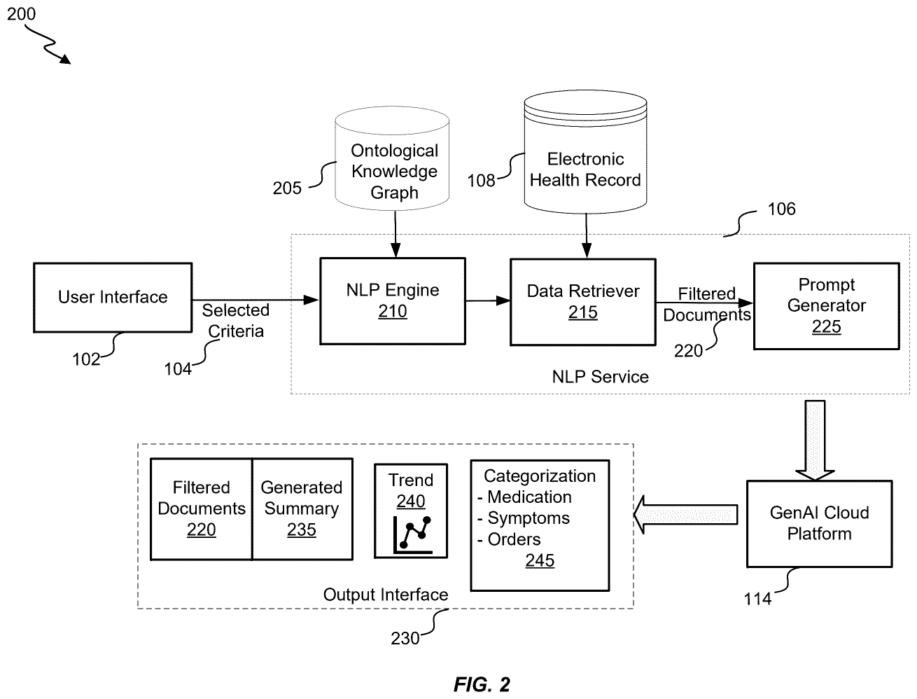

FIG. 2 shows an exemplary block diagram of a system to generate problem-oriented summary, trends, or categorized information based on the set of records of the subject and by using the GenAI model. The user interface 102 may be used to receive or access the message, email, or communication from the subject. The communication may include recent or past clinical conditions, for example, “The leg pain that I previously experienced seems to have improved significantly. However, I am now dealing with a persistent headache. Could you please prescribe medications for that.” In this exemplary communication or message, a past condition of leg pain (which is now improved) and a current condition of headache is mentioned. The conditions or clinical conditions (current or past) in the communication may be referred herein as ‘keywords’and can be extracted from the message or the communication using natural language processing techniques. The selected criteria 104 comprises of filters, choices, or selections made by the user by using the user interface 102 can be sent along with the message to the NLP service 106 for processing.

The NLP service 106 may be comprised of multiple components such as an NLP engine 210, a data retriever 215, and a prompt generator 225. The NLP engine 210 may be utilized to determine the context of the message, extracting one or more clinical conditions that may be present in the message. The NLP engine 210 can also perform a temporal analysis of the extracted clinical conditions (or keywords) to identify whether the target condition is a past issue or a current problem. For context analysis, the NLP engine 210 may break down the message to extract components including N-grams combinations to represent entities/phrases, noun phrases, themes, and Facets. The NLP engine 210 may utilize one or more algorithms or models to extract keywords or clinical conditions, for example TextRank algorithm can be used to understand the structure and content with the help of tokenization, building a graph representation, and ranking words based on their importance in the message. In some embodiments, the keyword extraction through the NLP engine 210 may utilize different models including and are not limited to, KeyBERT (leverages bidirectional encoder representations from transformers—BERT embeddings), yet another keyword extractor (YAKE—extracts words based on context and relevance), or rapid automatic keyword extraction (RAKE). After extraction of the keywords or determination of the context and intent of the message, the NLP engine 210 may further process the keywords (clinical conditions) or the context using the ontological knowledge graph 205.

The ontological knowledge graph 205 is an organizational graph representing an interconnected network of various terms within a specific domain (e.g., clinical domain) and may have three main components such as classes or entities, relationships, and their attributes. The ontological knowledge graph 205 may include inter-relationships of plurality of terms. The ontological knowledge graph 205 may utilize standard terminologies (e.g., SNOMED-CT, LOINC, ICD-10, and unified medical language system—UMLS), to define medical terms, hierarchies, and semantic relationships and based on information from medical databases, research articles, patient records, and clinical trials. The user may query precisely and find reasoning about medical concepts, drug discovery, disease associations, and personalized medicine. The ontological knowledge graph 205 may also include relationships between clinical and non-clinical terms. Exemplary sources for the clinical terms may include but are not limited to: SNOMED-CT for recording diagnoses, symptoms, and procedures; LOINC for assigning vocabulary codes to laboratory tests and other health-related data; ICD to use alphanumeric codes for diseases. In some instances, the non-clinical terms may represent the keywords (clinical conditions) extracted from the message. The NLP engine 210 may utilize the extracted keywords, or the context to identify related one or more clinical concepts (e.g., lab tests, symptoms, medications, diseases etc.) using the ontological knowledge graph 205. The ontological knowledge graph 205 may further include lexical variants of a non-clinical term. Examples of lexical variants of an extracted clinical condition headache may include tension headache, migraine, or cluster headache.

The NLP engine 210 may provide detailed and comprehensive ontologies or the one or more clinical concepts for the keywords (or clinical conditions), for example, related terminologies, diseases, symptoms, medications, and dosages. In some instances, the NLP engine 210 can be a model that is pre-trained on massive text corpora by using powerful hardware infrastructure, such as high-performance graphical processing units (GPUs) or TPUs. In some other instances, the NLP engine 210 may represent a model that is trained using large-scale datasets at first and then may be fine-tuned for specific tasks. Once trained or pre-trained, the NLP engine 210 can be deployed in different ways such as a cloud service, or as a computer program on the user device. The NLP engine 210 may provide the one or more clinical concepts, keywords, and the selected criteria 104 to the data retriever 215.

The data retriever 215 may utilize the one or more clinical concepts, keywords, or the selected criteria 104 to perform search for related records or documents in the database or the EHR 108. The data retriever 215 may search through the records of the subject and extract documents/notes, laboratory reports, historical data, clinical findings, and medications data related with the one or more clinical concepts, keywords, or the selected criteria 104. The data retriever 215 may refer to a process of filtering through a record pool (e.g., the EHR 108) and narrowing down to the set of records that match the context of the message (or communication) from the subject or the selected criteria 104 chosen by the user. The data retriever 215 may be comprised of simple keyword-based search and may use metadata to select documents based on the selected criteria 104 by the user. In some instances, document filtering in the data retriever 215 may be based on collaborative filtering, content-based filtering, or hybrid filtering. In some embodiments, the data retriever 215 may be implemented using vector-based approaches, for example, vector space models (VSM) that represent documents as vectors in a high-dimensional space and find cosine similarity between each dimension with target ontological terms (e.g., clinical concepts, keywords or clinical conditions, etc.). The vector-based approaches may further include latent semantic analysis (LSA to reduce the dimensionality of vector space while conserving the semantic information), Doc2Vec, FastText, and BERT to yield document filtering based on semantic relationships, context, and the like. The output of the data retriever 215 can be filtered documents 220. The filtered documents 220 may include records extracted from the EHR 108 or the database by the data retriever 215.

The prompt generator 225 may utilize the NLP techniques to generate prompts for the GenAI model of the GenAI cloud platform 114. In some embodiments, the prompt generator 225 may further create an inference request by combining the prompt with the set of records or the identifiers of the set of records. The prompt generator 225 may adjust or shape the behavior and output of the GenAI model through prompts. In some instances, the prompt generator 225 may include prompt tuning manually or automatically based on the GenAI model response (or the output) and the performance metrics. Techniques such as reinforcement learning, gradient-based optimizations and the like can be used for prompt tuning. According to some aspects of the present disclosure, the prompt generator 225 may further provide configuration settings, for example, the prompt, identity (ID) of the model to use, number of generated texts as ‘numGenerations’, the maximum number of tokens to predict per text generation as ‘maxTokens’, and the like.

The NLP service 106 may be further comprised of the REST based service. The REST based service may process (e.g., extract contents) the set of records retrieved by the data retriever 215 and may call the GenAI service 118 API of the GenAI cloud platform 114 with appropriate credentials. The REST based service may facilitate efficient communication and interaction between the user application (e.g. the user interface 102) and servers of the GenAI cloud platform 114 through the network 112.

The GenAI cloud platform 114 may refer to an artificial intelligence (AI)-based system capable of generating content based on the patterns learned from the training data or provided data. The GenAI cloud platform 114 may integrate text-based services, for example large language models such as GPTs, Grok, Gemini, Llama etc. The RAG based GenAI model may retrieve facts or information from data sources (e.g., the EHR 108) or the provided data (e.g., the set of records) and may reduce risk of sensitive data leakage. The GenAI cloud platform 114 can be used to analyze the set of records and to generate the problem-oriented summary.

The output of the GenAI cloud platform 114 may include a generated summary 235, a trend 240, and a categorized information 245. In some instances, the output may also include prescription suggestion based on the temporal analysis of subject data from previous encounters or visits and using comparative analysis with past prescriptions from other physicians. The output of the GenAI model may be displayed or accessed on the output interface 230. The output interface 230 can be a part or subset of the user interface 102 or can be a separate interface e.g., another GUI page to display the results.

The categorized information 245 may comprise different sections such as vitals, medications, symptoms, orders, prescriptions, or diagnoses. provide the user with information about various categories of retrieved and summarized data. The different sections of the categorized information 245 may display information extracted from the set of records of the subject. In some instances, the categorized information 245 may indicate diseases that are improved and the diseases which may be present, persistent, or encountered recently. The trend 240 may represent change in symptoms severity over time, or change in biomarkers or markers in lab tests, for example, variations in serum creatinine level, platelets count etc. based on the records of lab tests. The trend 240 may facilitate in better understanding of clinical conditions, accurate diagnoses by the user (or clinician), or to analyze efficacy or current therapies or medications. The generated summary 235 can be displayed on the output interface 230 alongside the filtered documents 220. The user can utilize this side-by-side comparison for better readability, editing or modifying the generated summary 235. Afterwards, in some instances, the user may approve and save the generated summary 235 (or the problem-oriented summary) in the EHR 108.

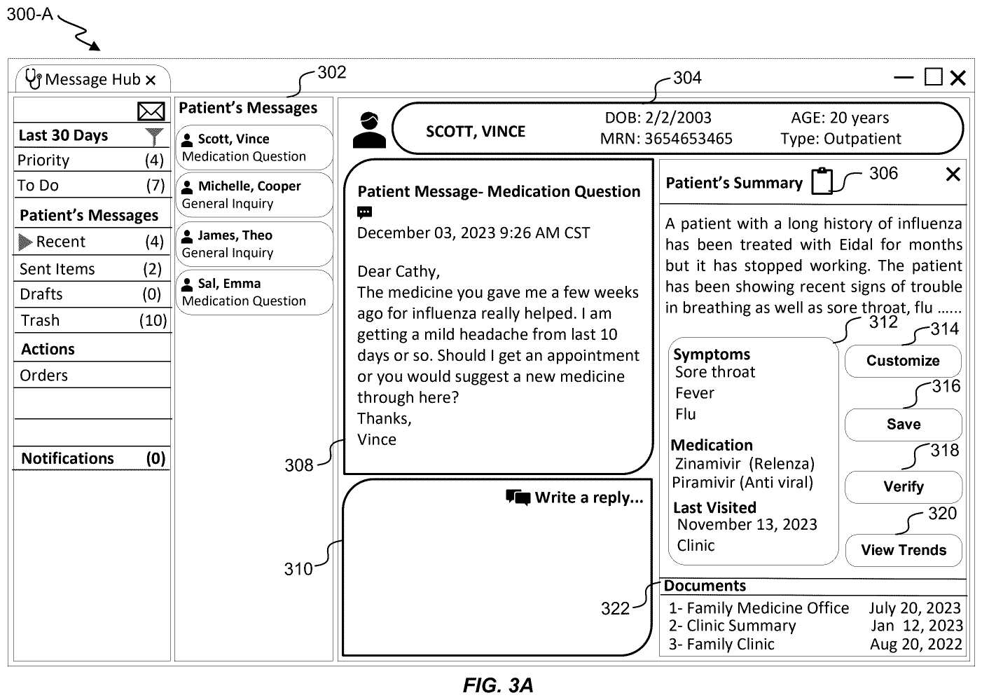

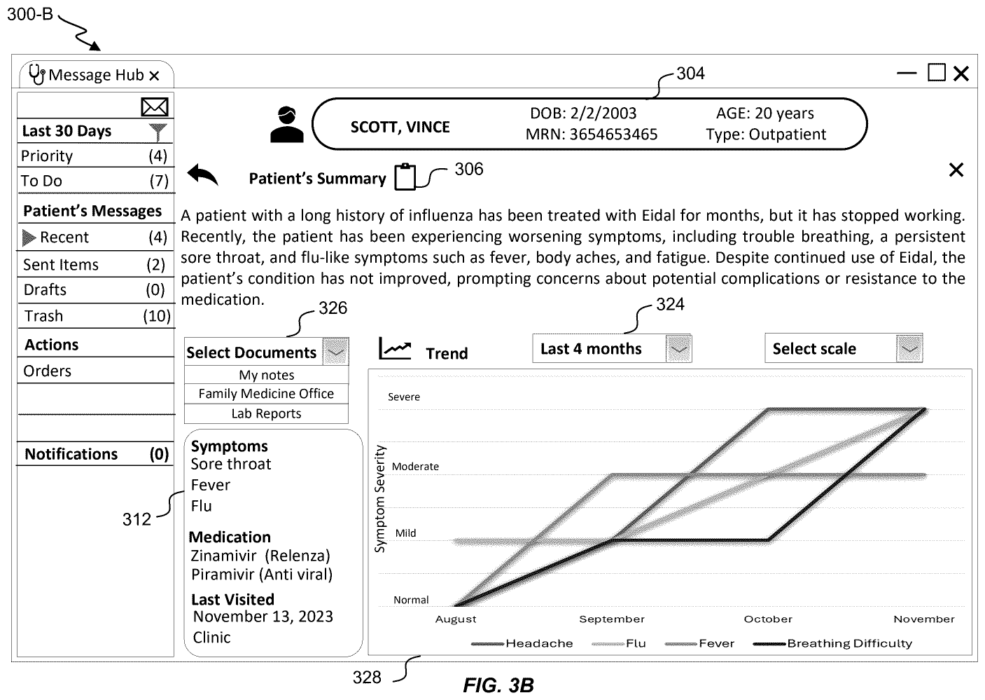

FIG. 3A shows an illustrative example of a first interface 300-A of the user interface 102 of an application that can be run on the user device. The application can be a dedicated application running on a computing device of the user, web-app, or a cloud-app. The first interface 300-A illustrates a message hub on the user interface 102 which may serve as a mode of communication between the user and the subject. On the first interface 300-A, the user can reach out to the subject, get updates on health conditions or address queries of the subject. A message panel 302 (or inbox) may include received messages from one or more subjects with respective names along with reason or subject of the message such as ‘General Inquiry’ or ‘Medication Question’. Upon selecting or opening a message from the message panel 302, one or more panels may appear, or the one or more panels may be already displayed on the first interface 300-A even before opening the message. The one or more panels may include a subject details section 304, a summary section 306, a message section 308, and a reply section 310. The one or more panels may be populated based on the subject details.

The subject details section 304 of the first interface 300-A may be utilized to display the details or personal details of the subject. The personal details of the subject may include subject name, date of birth, age, a type of patient such as ‘outpatient’ or ‘inpatient’, a profile picture, and a subject record number. In the message section 308, the time at which the message received can be shown followed by the contents or text of the message. This is followed by the reply section 310 in which the user can write back a suggestion or a prescription after reviewing or analyzing the summary section 306 and/or the categorized information 245 about the condition of the subject.

The summary section 306 of the first interface 300-A may display the generated summary 235 based on the filtered documents 220 (or the set of records) that is generated by using the GenAI service 118 or the GenAI cloud platform 114. The filtered documents 220 retrieved from the EHR 108 through the data retriever 215 can be shown in the form of a list in a document list 322 on the first interface 300-A. The documents or records in the document list 322 can be prioritized based on their dates and recent ones can be shown at the top of the document list 322. The summary section 306 is followed by a category segment 312 which may display the categorized information 245 such as recently occurring symptoms related to the problem being stated in the message section 308, along with the related prescribed medication, and the date of last visit to the clinic. The summary section 306 can be customized, verified and saved by the user upon selecting a customize option 314, a save option 316, and a verify option 318 respectively.

In some other instances, the problem-oriented summary may be automatically generated upon selecting a particular subject message from the message panel 302. In case the generated summary 235 lacks some points or details, the user can easily edit, remove content, or add some details according to his preferences by selecting the customize option 314. In some other instances, when the user is satisfied with the problem-oriented summary, the user can simply save it to the EHR 108 or the database by selecting the save option 316. In addition, the user can also view recent symptom trends related to the disease using a view trends option 320. On selecting the view trends option 320, a second interface 300-B can be displayed on the message hub.

FIG. 3B shows an illustrative example of the second interface 300-B of the user interface 102 of the application that can be run on the user device. The second interface 300-B may include the subject details section 304, the summary section 306, a drop-down menu 326, the category segment 312, a time duration filter 324, and a trend section 328. The drop-down menu 326 may be used to select specific documents from the document list 322 (or the filtered documents 220 or the set of records) in the first interface 300-A. A trend can be generated based on the selected specific documents and can be depicted using the trend section 328. Moreover, the time duration filter 324 such as ‘Last year’, ‘Last 4 months’, ‘Last month’ or ‘Encounter-wise’ may be applied to limit the selected documents for the trend section 328.

In some instances, the user may select one or more selection criteria at first (e.g., time duration, encounter type, record type etc.) and then the problem-oriented summary may be generated automatically on opening the message of the subject. The one or more selection criteria can be selected by the user through the time duration filter 324 or the drop-down menu 326. The drop-down menu 326 may include options such as ‘encounter-wise’, ‘lab reports’ or ‘author-wise notes’.

The trend section 328 may illustrate temporal variation in severity level of symptoms associated with the disease, problem, clinical condition, or the ailment mentioned by the subject in the message section 308. The trend section 328 may be generated based on part or all the filtered documents 220 appearing in the documents list 322 or the trend can be based upon the documents appearing at the top of the documents list 322.

In some instances, the user can select whether to incorporate the records of the subject that are not authored by the user, for example, medical suggestions from other clinics in addition to the records authored by the user. This may be effective in situations when the subject may not be regularly visiting a specific clinic and may visit different healthcare facilities at different times. In such cases, records of the subject at some clinic or authored by the user may not provide complete picture of the health condition of the subject. Consequently, the trend section 328 can be generated using all the documents appearing in the documents list 322.

In some instances, the user can have regular information on the conditions of the subject and wants to see the trends based on personal notes, then the user may select the option of ‘My notes’ from the drop-down menu 326. Furthermore, the user can select the appropriate time limit from the time duration filter 324 in order to focus on recent data of a long-term condition.

The trend section 328 can be used to display multiple symptoms or health indicators related to the disease on four levels: ‘normal’, ‘mild’, ‘moderate’, and ‘severe’. This categorization is achieved by using large language models (LLMs) or the GenAI model to analyze and interpret the data from lab reports, notes, records, or documents. The GenAI model may process the raw data, identify patterns, and assign severity levels to each symptom based on predefined criteria. Using this scale for the symptoms severity can provide a collective visual of symptoms corresponding to an individual or the subject on a single graph. This may further simplify the assessment of multiple symptoms by the user and to decide on what to prescribe next. By reviewing the recently occurring symptoms (e.g., from the received message), medications currently taken or other data from the category segment 312, along with the history of symptoms severity in the trend section 328, the user can make an informed decision about the subject.

In some other instances, the user may want to review recent trends of vitals or biomarkers, such as platelets count, red blood cells or white blood cells count, hemoglobin level from the lab reports of the subject. By selecting the lab reports option from the drop-down menu 326, the user can view a trend that shows the levels of the vitals or biomarkers over a specific period. Additionally, the time duration filter 324 can be utilized to obtain the trends over various periods, such as the past year or the last few months. Therefore, the user can easily and accurately analyze deviations in vitals or biomarkers within the chosen timeframe.

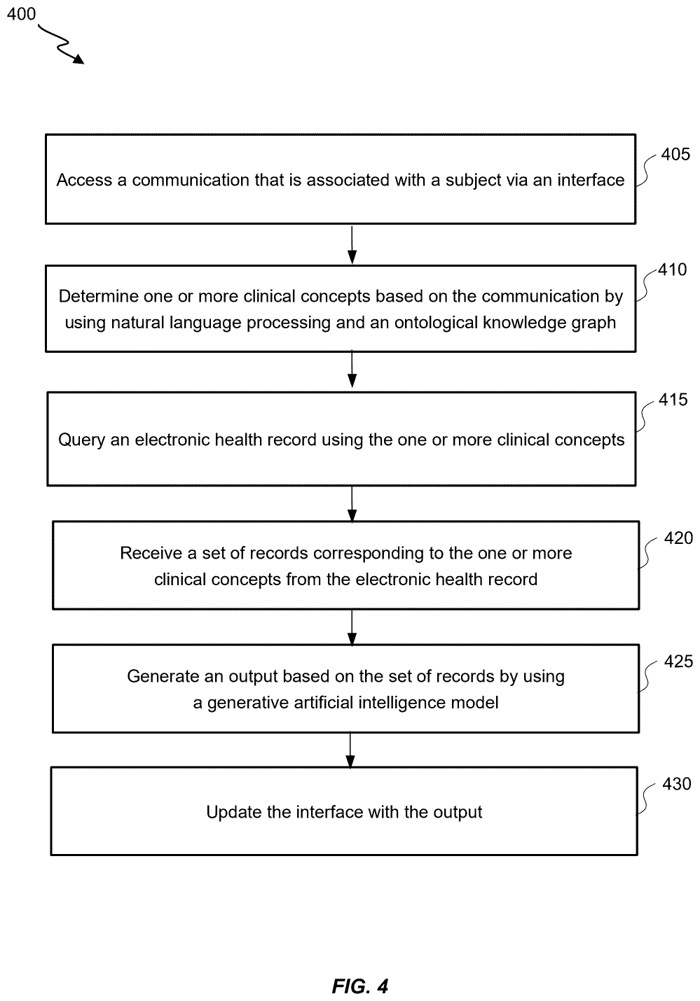

FIG. 4 shows an example flowchart of a system to generate a problem-oriented summary, trends, or categorized information based on the set of records of the subject in accordance with some embodiments of the present disclosure. The blocks in flowchart 400 are illustrated in a specific order, while the order can be modified, for example, some blocks may be performed before other, and some blocks may be performed simultaneously. The blocks can be performed by hardware or software or a combination thereof. The process at block 405 may include accessing a communication, an email, or a message that is associated with a subject via an interface such as user interface 102.

One or more clinical concepts may be determined based on the communication by using natural language processing and the ontological knowledge graph, at block 410. The ontological knowledge graph may define semantics, constraints, and relationships between a plurality of terms. The process at block 415 may include querying an electronic health record using the one or more clinical concepts. In some instances, one or more selection criteria may be received from a user device by using the user interface 102. The one or more selection criteria may comprise one or more filters corresponding to a time interval, an encounter type, a record type, or an author of a record. The electronic health record may be queried using the one or more clinical concepts and the one or more selection criteria.

A set of records corresponding to the one or more clinical concepts may be received from the electronic health record in response to the query, at block 420. An inference request can be generated for the GenAI model. The inference request may be comprised of a prompt and the set of records, or the prompt and an identifier for each record of the set of records.

An output based on the set of records may be generated by using the GenAI model, at block 425. The GenAI model may include a retrieval-augmented generation (RAG) model, a fine-tuned transformer model, or a domain-specific model that is trained on domain-specific data. The GenAI model may be configured to generate an augmented context by combining a prompt with the set of records. The set of records may provide additional information to the GenAI model and were not included in training data of the GenAI model. An inference request can be executed using the GenAI model and the output may be generated based on the augmented context by using the GenAI model.

The output may be comprised of a summary of the set of records, a trend, or a categorization of information of the set of records. The summary can be a problem-oriented summary, or a disease-oriented summary based on the set of records. The trend may represent variation in severity of symptoms and can be obtained by temporal analysis of data from the set of records. Moreover, the output may comprise categorized information of the set of records corresponding to a plurality of sections. The plurality of sections may include symptoms, medications, lab tests, and diagnosis. Finally, at block 430, the interface (e.g., the user interface 102) may be updated with the output. The user may review, edit, sign, or save the output (e.g., the summary) and/or may make informed decisions about the subject.

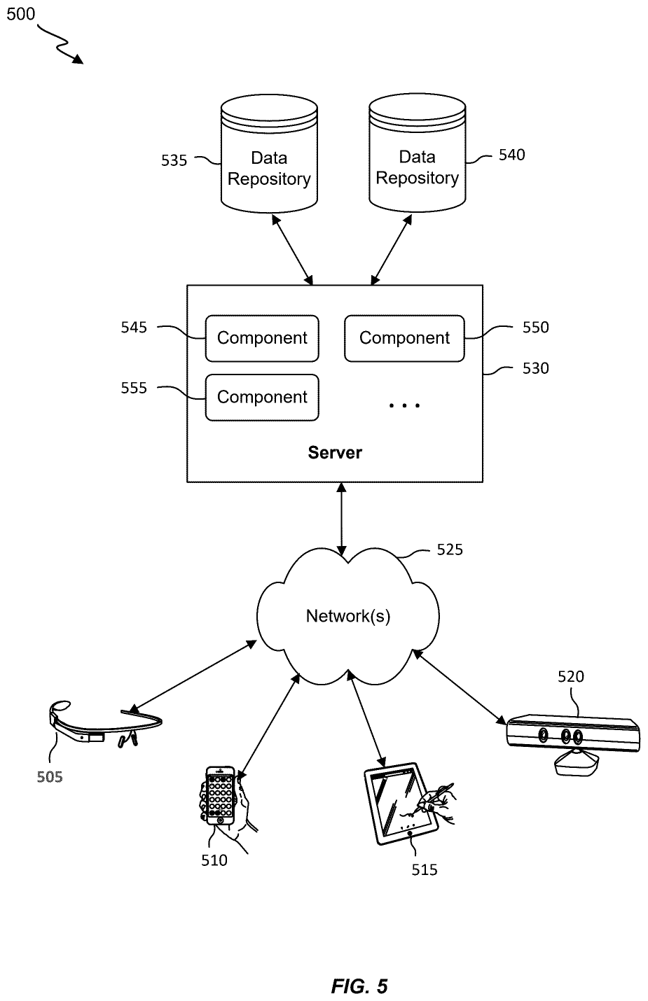

FIG. 5 illustrates a simplified diagram of an example distributed system 500 for a cloud hosting a GenAI platform. In the illustrated example, the distributed system 500 includes one or more client computing devices 505, 510, 515, and 520, coupled to a the server 530 via one or more communication networks 525. The clients computing devices 505, 510, 515, and 520 may be configured to execute one or more applications interact with the server 530 to access and utilize the GenAI platform securely integrated within a cloud environment, such as Oracle cloud integrated with Cohere. Within this framework, the server 530 is configured to host and manage a range of services or software applications, facilitating seamless integration and operation of the GenAI platform.

In various aspects, the server 530 may extend its capabilities to encompass additional services or software applications. These services may span both virtual and non-virtual environments, enabling a comprehensive and adaptable infrastructure for securely deploying GenAI solutions within the cloud ecosystem. In some respects, these services may be offered as web-based or cloud services, such as under a Software as a Service (SaaS) model to the users of the client computing devices 505, 510, 515, and/or 520. Users operating the client computing devices 505, 510, 515, and/or 520 may in turn utilize one or more client applications to interact with the server 530 to utilize the services provided by these components. Furthermore, client computing devices 505, 510,515, and/or 520 may in turn utilize one or more client applications to initiate and manage specific tasks or analyses within the GenAI platform.

In the configuration depicted in FIG. 5, the server 530 may include one or more components 545, 550 and 555 that implement the functions performed by the server 530. These components may include software components that may be executed by one or more processors, hardware components, or combinations thereof. It should be appreciated that various system configurations are possible, which may differ from distributed system 500. The example shown in FIG. 5 is thus one example of a distributed system for implementing an example system and is not intended to be limiting.

Users may initiate requests for the GenAI platform through client computing devices 505, 510, 515, and/or 520 for inference or other machine-learning tasks. A client device may provide an interface that enables a user of the client device to interact with the GenAI platform. The client device may also output information to the user via this interface. Although FIG. 5 depicts only four client computing devices, any number of client computing devices may be supported providing scalability and accessibility within the integrated GenAI platform on the cloud.

The client devices may include various types of computing systems, such as portable handheld devices, general purpose computers, such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems, such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone®), tablets (e.g., iPad®), personal digital assistants (PDAs), and the like. Wearable devices may include Google Glass® head mounted display, and other devices. Gaming systems may include various handheld gaming devices, Internet-enabled gaming devices (e.g., a Microsoft Xbox® gaming console with or without a Kinect® gesture input device, Sony PlayStation® system, various gaming systems provided by Nintendo®, and others), and the like. The client devices may be capable of executing various applications, such as various Internet-related apps, communication applications (e.g., E-mail applications, short message service (SMS) applications) and may use various communication protocols.

Network(s) 1025 may be any type of network familiar to those skilled in the art that can support data communications using any of a variety of available protocols, including without limitation TCP/IP (transmission control protocol/internet protocol), SNA (systems network architecture), IPX (internet packet exchange), AppleTalk®, and the like. Merely by way of example, network(s) 1025 can be a local area network (LAN), networks based on ethernet, token-ring, a wide-area network (WAN), the internet, a virtual network, a virtual private network (VPN), an intranet, an extranet, a public switched telephone network (PSTN), an infra-red network, a wireless network (e.g., a network operating under any of the institute of electrical and electronics (IEEE) 1002.11 suite of protocols, Bluetooth®, and/or any other wireless protocol), and/or any combination of these and/or other networks.

The server 530 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. The server 530 can include one or more virtual machines running virtual operating systems, or other computing architectures involving virtualization, such as one or more flexible pools of logical storage devices that can be virtualized to maintain virtual storage devices for the server. In various aspects, the server 530 may be adapted to run one or more services or software applications that provide the functionality described in the foregoing disclosure.

The computing systems in the server 530 may run one or more operating systems including any of those discussed above, as well as any commercially available server operating system. The server 530 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

Distributed system 500 may also include one or more data repositories 535, 540. Data repositories 535, 540 may reside in many locations. For example, a data repository used by the server 530 may be local to server 1030 or may be remote from the server 530 and in communication with the server 530 via a network-based or dedicated connection. Data repositories 535, 540 may be of different types. In some instances, a data repository used by the server 530 may be a database, for example, a relational database, such as databases provided by Oracle Corporation® and other vendors. One or more of these databases may be adapted to enable storage, update, and retrieval of data to and from the database in response to structured query language (SQL)-formatted commands. In some aspects, one or more data repositories 535, 540 may also be used by applications to store application data. The data repositories used by applications may be of different types, such as, for example, a key-value store repository, an object store repository, or a general storage repository supported by a file system.

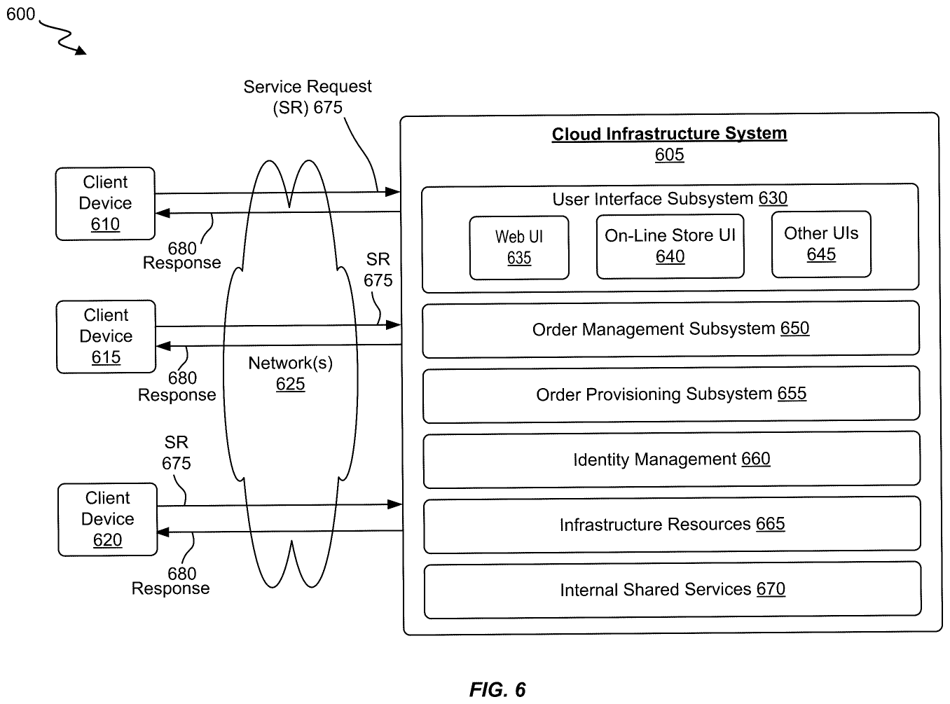

FIG. 6 is a simplified block diagram of a cloud-based system environment in which various services of the server 530 of FIG. 5 may be offered as cloud services, in accordance with certain aspects. In the illustrative example depicted in FIG. 6, cloud infrastructure system 605 may provide one or more cloud services that may be requested by users using one or more client devices 610, 615, and 620. Cloud infrastructure system 605 may comprise one or more computers and/or servers that may include those described for server 530. The computers in cloud infrastructure system 605 may be organized as general-purpose computers, specialized server computers, server farms, server clusters, or any other appropriate arrangement and/or combination.

Network(s) 625 may facilitate communication and exchange of data between client devices 610, 615, and 620 and cloud infrastructure system 605. Network(s) 625 may include one or more networks. The networks may be of the same or different types. Network(s) 625 may support one or more communication protocols, including wired and/or wireless protocols, for facilitating the communications.

The illustrative example depicted in FIG. 6 is only one example of a cloud infrastructure system 605 and is not intended to be limiting. It should be appreciated that, in some other aspects, cloud infrastructure system 605 may have more or fewer components than those depicted in FIG. 6, may combine two or more components, or may have a different configuration or arrangement of components. For example, although FIG. 6 depicts three client computing devices, any number of client computing devices may be supported in alternative aspects.

The term cloud service is generally used to refer to a service that is made available to users on demand and via a communication network, such as the internet by systems (e.g., cloud infrastructure system 605) of a service provider. Typically, in a public cloud environment, servers and systems that make up the cloud service provider's system are different from the client's own on-premises servers and systems. The cloud service provider's systems are managed by the cloud service provider. Clients can thus avail themselves of cloud services provided by a cloud service provider without having to purchase separate licenses, support, or hardware and software resources for the services. For example, a cloud service provider's system may host an application, and a user may, via a network 625 (e.g., the internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation® of Redwood Shores, California, such as middleware services, database services, Java cloud services, and others.

In certain aspects, cloud infrastructure system 605 may provide one or more cloud services using different models, such as under a Software as a Service (SaaS) model, a Platform as a Service (PaaS) model, an Infrastructure as a Service (IaaS) model, and others, including hybrid service models. Cloud infrastructure system 605 may include a suite of applications, middleware, databases, and other resources that enable provision of the various cloud services.

A SaaS model enables an application or software to be delivered to a client over a communication network like the Internet, as a service, without the client having to buy the hardware or software for the underlying application. For example, a SaaS model may be used to provide clients access to on-demand applications that are hosted by cloud infrastructure system 605. Examples of SaaS services provided by Oracle Corporation® include, without limitation, various services for human resources/capital management, client relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a client as a cloud service to provide elastic compute and storage capabilities. Various IaaS services are provided by Oracle Corporation®.

A PaaS model is generally used to provide, as a service, platform and environment resources that enable clients to develop, run, and manage applications and services without the client having to procure, build, or maintain such resources. Examples of PaaS services provided by Oracle Corporation® include, without limitation, Oracle Java Cloud Service (JCS), Oracle Database Cloud Service (DBCS), data management cloud service, various application development solutions services, and others.

Cloud services are generally provided on an on-demand self-service basis, subscription-based, elastically scalable, reliable, highly available, and secure manner. For example, a client, via a subscription order, may order one or more services provided by cloud infrastructure system 605. Cloud infrastructure system 605 then performs processing to provide the services requested in the client's subscription order. Cloud infrastructure system 605 may be configured to provide one or even multiple cloud services.

Cloud infrastructure system 605 may provide cloud services via different deployment models. In a public cloud model, cloud infrastructure system 605 may be owned by a third-party cloud services provider and the cloud services are offered to any general public client, where the client can be an individual or an enterprise. In certain other aspects, under a private cloud model, cloud infrastructure system 605 may be operated within an organization (e.g., within an enterprise organization) and services provided to clients that are within the organization. For example, the clients may be various departments of an enterprise, such as the Human Resources department, the payroll department, etc. or even individuals within the enterprise. In certain other aspects, under a community cloud model, the cloud infrastructure system 605 and the services provided may be shared by several organizations in a related community. Various other models, such as hybrids of the above-mentioned models may also be used.

Client devices 610, 615, and 620 may be of several types (such as client computing devices 505, 510, 515, and 520 depicted in FIG. 5) and may be capable of operating one or more client applications. A user may use a client device to interact with cloud infrastructure system 605, such as to request a service provided by cloud infrastructure system 605. For instance, a user might employ a client device to execute real-time data querying operations within the cloud. A GenAI client may use a client device, such as a laptop to interact with the GenAI platform integrated within cloud infrastructure system. The client may request GPU-accelerated computing instances of the cloud for training deep learning models. The cloud may provide the necessary resources, and the GenAI client may monitor and manage the training process through the laptop. Upon completion, the client may retrieve the trained models and results.

In certain aspects, to facilitate efficient provisioning of these resources for supporting the various cloud services provided by cloud infrastructure system 605 for different clients, the resources may be bundled into sets of resources or resource modules. Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 605 may comprise multiple subsystems. These subsystems may be implemented in software, or hardware, or combinations thereof. As depicted in FIG. 6, the subsystems may include a user interface subsystem 630 that enables users or clients of cloud infrastructure system 605 to interact with cloud infrastructure system 605. User interface subsystem 630 may include various interfaces, such as a web user interface 635, an online store interface 640 where cloud services provided by cloud infrastructure system 605 are advertised and are purchasable by a consumer, and other interfaces 645. For example, a client may, using a client device, request (service request 675) one or more services provided by cloud infrastructure system 605 using one or more of interfaces 635, 640, and 645. For example, a client may access the online store, browse cloud services offered by cloud infrastructure system 605, and place a subscription order for one or more services offered by cloud infrastructure system 605 that the client wishes to subscribe to. The service request may include information identifying the client and one or more services that the client desires to subscribe to. For example, a client may place a subscription order for a Chabot related service offered by cloud infrastructure system 605. As part of the order, the client may provide information identifying for input (e.g., utterances).

In certain aspects, such as the illustrative example depicted in FIG. 6, cloud infrastructure system 605 may comprise an order management subsystem (OMS) 650 that is configured to process the new order. As part of this processing, OMS 650 may be configured to: create an account for the client, if not done already; receive billing and/or accounting information from the client that is to be used for billing the client for providing the requested service to the client; verify the client information; upon verification, book the order for the client; and orchestrate various workflows to prepare the order for provisioning.

Once properly validated, OMS 650 may then invoke the order provisioning subsystem (OPS) 655 that is configured to provision resources for the order including processing, memory, and networking resources. The provisioning may include allocating resources for the order and configuring the resources to facilitate the service requested by the client order. The way resources are provisioned for an order and the type of the provisioned resources may depend upon the type of cloud service that has been ordered by the client. For example, according to one workflow, OPS 655 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the requesting client for providing the requested service.

Cloud infrastructure system 605 may itself internally use services 670 that are shared by different components of cloud infrastructure system 605 and which facilitate the provisioning of services by cloud infrastructure system 605. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like. As depicted in the illustrative example in FIG. 6, cloud infrastructure system 605 may include infrastructure resources 665 that can be utilized for facilitating the provision of various cloud services offered by cloud infrastructure system 605. Infrastructure resources 665 may include, for example, processing resources, storage or memory resources, networking resources, and the like. Cloud infrastructure system 605 may send a response or notification 680 to the requesting client to indicate when the requested service is now ready for use. In some instances, information (e.g., a link) may be sent to the client that enables the client to start using and availing the benefits of the requested services.

Cloud infrastructure system 605 may provide services to multiple clients in parallel. Cloud infrastructure system 605 may store information for these clients, including possibly proprietary information. In certain aspects, cloud infrastructure system 605 comprises an identity management subsystem (IMS) 660 that is configured to manage client's information and provide the separation of the managed information such that information related to one client is not accessible by another client. IMS 660 may be configured to provide various security-related services, such as identity services, such as information access management, authentication and authorization services, services for managing client identities and roles and related capabilities, and the like.

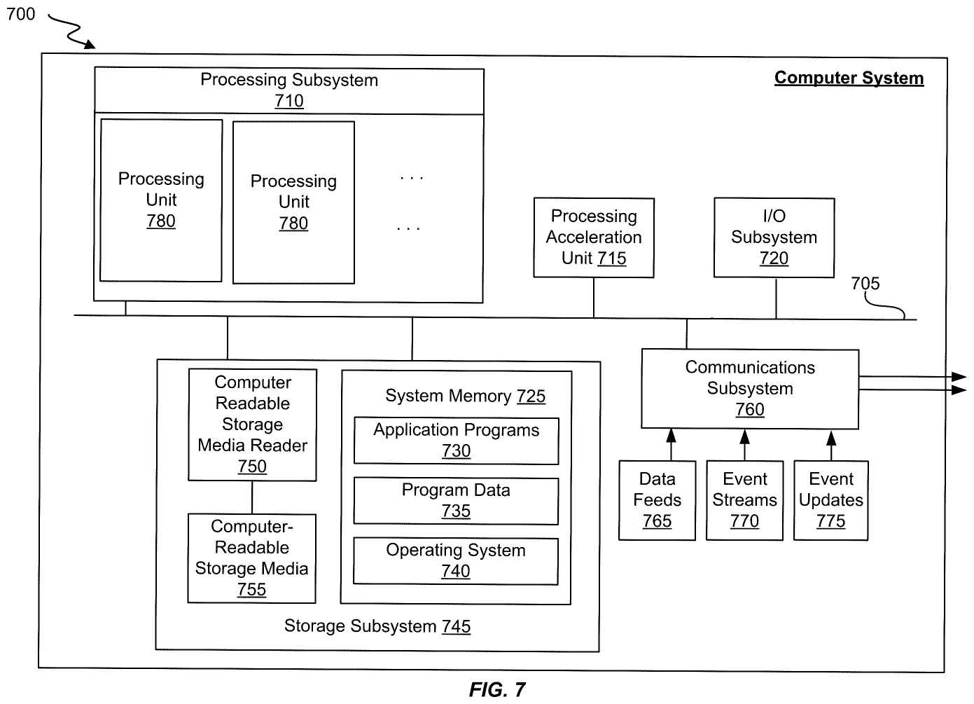

FIG. 7 illustrates an exemplary computer system 700 that may be used to implement certain aspects of the present disclosure. For example, a computer system 700 may facilitate the integration of a GenAI platform with the cloud by provisioning and configuring resources, managing data, implementing security measures, monitoring performance, and enabling scalability. It may serve as the foundational infrastructure, enabling seamless deployment and operation of AI applications within the cloud environment while providing flexibility and scalability to adapt to changing computational demands efficiently. In some aspects, computer system 700 may be used to implement various servers as described above. As shown in FIG. 7, computer system 700 may include various subsystems including a processing subsystem 710 that communicates with a few other subsystems via a bus subsystem 705. These other subsystems may include a processing acceleration unit 715, an I/O subsystem 720, a storage subsystem 745, and a communications subsystem 760. Storage subsystem 745 may include non-transitory computer-readable storage media including storage media 755 and a system memory 725.

Bus subsystem 705 provides a mechanism for letting the various components and subsystems of computer system 700 communicate with each other as intended. Although bus subsystem 705 is shown schematically as a single bus, alternative aspects of the bus subsystem may utilize multiple buses. Bus subsystem 705 may be any of several types of bus structures including a memory bus or memory controller, a peripheral bus, a local bus using any of a variety of bus architectures, and the like. For example, such architectures may include an industry standard architecture (ISA) bus, micro channel architecture (MCA) bus, enhanced ISA (EISA) bus, video electronics standards association (VESA) local bus, and peripheral component interconnect (PCI) bus, which can be implemented as a Mezzanine bus manufactured to the IEEE P1386.1 standard, and the like.

Processing subsystem 710 controls the operation of computer system 700 and may comprise one or more processors, application specific integrated circuits (ASICs), or field programmable gate arrays (FPGAs). The processors may include single core or multicore processors. The processing resources of computer system 700 can be organized into one or more processing units 780, 780, etc. A processing unit may include one or more processors, one or more cores from the same or different processors, a combination of cores and processors, or other combinations of cores and processors. In some aspects, processing subsystem 710 can include one or more special purpose co-processors, such as graphics processors, digital signal processors (DSPs), or the like. In some aspects, some or all of the processing units of processing subsystem 710 can be implemented using customized circuits, such as application specific integrated circuits (ASICs), or field programmable gate arrays (FPGAs).

In some aspects, the processing units in processing subsystem 710 can execute instructions stored in system memory 725 or on computer readable storage media 755. In various aspects, the processing units can execute a variety of programs or code instructions and can maintain multiple concurrently executing programs or processes. At any given time, some, or all of the program code to be executed can be resident in system memory 725 and/or on computer-readable storage media 755 including potentially on one or more storage devices. Through suitable programming, processing subsystem 710 can provide various functionalities described above. In instances where computer system 700 is executing one or more virtual machines, one or more processing units may be allocated to each virtual machine.

In certain aspects, a processing acceleration unit 715 may optionally be provided for performing customized processing or for off-loading some of the processing performed by processing subsystem 710 to accelerate the overall processing performed by computer system 700.

I/O subsystem 720 may include devices and mechanisms for inputting information to computer system 700 and/or for outputting information from or via computer system 700. In general, use of the term input device is intended to include all possible types of devices and mechanisms for inputting information to computer system 700. User interface input devices may include, for example, a keyboard, pointing devices, such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices, such as the Microsoft Kinect® motion sensor that enables users to control and interact with an input device, the Microsoft Xbox® 360 game controller, devices that provide an interface for receiving input using gestures and spoken commands. User interface input devices may also include eye gesture recognition devices, such as the Google Glass® blink detector that detects eye activity (e.g., “blinking” while taking pictures and/or making a menu selection) from users and transforms the eye gestures as inputs to an input device (e.g., Google Glass®). Additionally, user interface input devices may include voice recognition sensing devices that enable users to interact with voice recognition systems (e.g., Siri® navigator) through voice commands.

Other examples of user interface input devices include, without limitation, three dimensional (3D) mice, joysticks or pointing sticks, gamepads and graphic tablets, and audio/visual devices, such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices, such as computed tomography, magnetic resonance imaging, position emission tomography, and medical ultrasonography devices. User interface input devices may also include, for example, audio input devices, such as MIDI keyboards, digital musical instruments, and the like.

In general, use of the term output device is intended to include all possible types of devices and mechanisms for outputting information from computer system 700 to a user or other computer. User interface output devices may include a display subsystem, indicator lights, or non-visual displays, such as audio output devices, etc. The display subsystem may be a cathode ray tube (CRT), a flat-panel device, such as that using a liquid crystal display (LCD) or plasma display, a projection device, a touch screen, and the like. For example, user interface output devices may include, without limitation, a variety of display devices that visually convey text, graphics, and audio/video information, such as monitors, printers, speakers, headphones, automotive navigation systems, plotters, voice output devices, and modems.

Storage subsystem 745 provides a repository or data store for storing information and data that is used by computer system 700. Storage subsystem 745 provides a tangible non-transitory computer-readable storage medium for storing the basic programming and data constructs that provide the functionality of some aspects. Storage subsystem 745 may store software (e.g., programs, code modules, instructions) that when executed by processing subsystem 710 provides the functionality described above. The software may be executed by one or more processing units of processing subsystem 710. Storage subsystem 745 may also provide a repository for storing data used in accordance with the teachings of this disclosure.

Storage subsystem 745 may include one or more non-transitory memory devices, including volatile and non-volatile memory devices. As shown in FIG. 7, storage subsystem 745 includes a system memory 725 and a computer-readable storage media 755. System memory 725 may include a number of memories including a volatile main random-access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system 700, such as during start-up, may typically be stored in the ROM. The RAM typically contains data and/or program modules that are presently being operated and executed by processing subsystem 710. In some implementations, system memory 725 may include multiple different types of memory, such as static random-access memory (SRAM), dynamic random-access memory (DRAM), and the like.

By way of example, and not limitation, as depicted in FIG. 7, system memory 725 may load application programs 730 that are being executed, which may include various applications, such as Web browsers, mid-tier applications, relational database management systems (RDBMS), etc., program data 735, and an operating system 740. By way of example, operating system 740 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems, such as iOS, Windows® Phone, Android® OS, BlackBerry® OS, Palm® OS operating systems, and others.

Computer-readable storage media 755 may store programming and data constructs that provide the functionality of some aspects. Computer-readable media 755 may provide storage of computer-readable instructions, data structures, program modules, and other data for computer system 700. Software (programs, code modules, instructions) that, when executed by processing subsystem 710 provides the functionality described above, may be stored in storage subsystem 745. By way of example, computer-readable storage media 755 may include non-volatile memory, such as a hard disk drive, a magnetic disk drive, an optical disk drive, such as a CD ROM, digital video disc (DVD), a Blu-Ray® disk, or other optical media. Computer-readable storage media 1255 may include, but is not limited to, Zip® drives, flash memory cards, universal serial bus (USB) flash drives, secure digital (SD) cards, DVD disks, digital video tape, and the like. Computer-readable storage media 755 may also include, solid-state drives (SSD) based on non-volatile memory, such as flash-memory based SSDs, enterprise flash drives, solid state ROM, and the like, SSDs based on volatile memory, such as solid state RAM, dynamic RAM, static RAM, dynamic random access memory (DRAM)-based SSDs, magneto resistive RAM (MRAM) SSDs, and hybrid SSDs that use a combination of DRAM and flash memory based SSDs.

In certain aspects, storage subsystem 745 may also include a computer-readable storage media reader 750 that can further be connected to computer-readable storage media 755. The computer-readable storage media reader 750 may receive and be configured to read data from a memory device, such as a disk, a flash drive, etc.

In certain aspects, computer system 700 may support virtualization technologies, including but not limited to virtualization of processing and memory resources. For example, computer system 700 may provide support for executing one or more virtual machines. In certain aspects, computer system 700 may execute a program, such as a hypervisor that facilitated the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 700. Accordingly, multiple operating systems may potentially be run concurrently by computer system 700.

Communications subsystem 760 provides an interface to other computer systems and networks. Communications subsystem 760 serves as an interface for receiving data from and transmitting data to other systems from computer system 700. For example, communications subsystem 760 may enable computer system 700 to establish a communication channel to one or more client devices via the Internet for receiving and sending information from and to the client devices. For example, the communication subsystem may be used to transmit a response to a user regarding the inquiry for a Chabot.

Communication subsystem 760 may support both wired and/or wireless communication protocols. For example, in certain aspects, communications subsystem 760 may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 802.XX family standards, or other mobile communication technologies, or any combination thereof), global positioning system (GPS) receiver components, and/or other components. In some aspects, communications subsystem 760 can provide wired network connectivity (e.g., Ethernet) in addition to or instead of a wireless interface.

Communication subsystem 760 can receive and transmit data in various forms. For example, in some aspects, in addition to other forms, communications subsystem 760 may receive input communications in the form of data feeds 765 such as structured and/or unstructured data feeds, event streams 770, event updates 775, and the like. For example, communications subsystem 760 may be configured to receive (or send) data feeds 765 in real-time from users of social media networks and/or other communication services, such as Twitter® feeds, Facebook® updates, web feeds, such as Rich Site Summary (RSS) feeds, and/or real-time updates from one or more third party information sources.

In certain aspects, communications subsystem 760 may be configured to receive data in the form of continuous data streams, which may include event streams 770 of real-time events and/or event updates 775, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 760 may also be configured to communicate data from computer system 700 to other computer systems or networks. The data may be communicated in various forms, such as structured and/or unstructured data feeds 765, event streams 770, event updates 775, and the like to one or more databases that may be in communication with one or more streaming data source computers coupled to computer system 700.

Computer system 700 can be one of various types, including a handheld portable device (e.g., an iPhone® cellular phone, an iPad® computing tablet, a personal digital assistant (PDA)), a wearable device (e.g., a Google Glass® head mounted display), a personal computer, a workstation, a mainframe, a kiosk, a server rack, or any other data processing system. Due to the ever-changing nature of computers and networks, the description of computer system 700 depicted in FIG. 7 is intended only as a specific example. Many other configurations having more or fewer components than the system depicted in FIG. 7 are possible. Based on the disclosure and teachings provided herein, a person of ordinary skill in art can appreciate other ways and/or methods to implement the various aspects.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed:

1. A computer-implemented method comprising:
accessing a communication that is associated with a subject via an interface;
determining one or more clinical concepts based on the communication by using natural language processing and an ontological knowledge graph, wherein the ontological knowledge graph defines semantics, constraints, and relationships between a plurality of terms;
querying an electronic health record using the one or more clinical concepts;
receiving, in response to the query, a set of records corresponding to the one or more clinical concepts from the electronic health record;
generating an output based on the set of records by using a generative artificial intelligence (GenAI) model, wherein the output comprises a summary of the set of records, a trend, or a categorization of information of the set of records; and
updating the interface with the output.

2. The computer-implemented method of claim 1, further comprising:
generating an inference request for the GenAI model, wherein:
the inference request comprises a prompt and the set of records, or
the inference request comprises the prompt and an identifier for each record of the set of records.

3. The computer-implemented method of claim 1, wherein generating the output based on the set of records by using the GenAI model further includes:
executing an inference request using the GenAI model, wherein the GenAI model is configured to generate an augmented context by combining a prompt with the set of records, and wherein the set of records were not included in training data of the GenAI model; and
generating the output based on the augmented context by using the GenAI model.

4. The computer-implemented method of claim 1, wherein the GenAI model includes a retrieval-augmented generation (RAG) model, a fine-tuned transformer model, or a domain-specific model that is trained on domain-specific data.

5. The computer-implemented method of claim 1, further comprising:
receiving one or more selection criteria from a user device, wherein the one or more selection criteria comprises one or more filters corresponding to a time interval, an encounter type, a record type, or an author of a record;
querying the electronic health record using the one or more clinical concepts and the one or more selection criteria;
receiving, in response to the query, a second set of records corresponding to the one or more clinical concepts and the one or more selection criteria; and
generating a second output based on the second set of records by using the GenAI model.

6. The computer-implemented method of claim 1, wherein the output comprises a problem-oriented summary or a disease-oriented summary based on the set of records.

7. The computer-implemented method of claim 1, wherein the output comprises the trend of symptoms by using temporal analysis of data from the set of records.

8. The computer-implemented method of claim 1, wherein the output comprises categorized information of the set of records corresponding to a plurality of sections, wherein the plurality of sections includes symptoms, medications, lab tests, and diagnosis.

9. A system comprising:
one or more data processors; and
a non-transitory computer readable storage medium containing instructions which, when executed on the one or more data processors, cause the one or more data processors to perform a set of operations including:
accessing a communication that is associated with a subject via an interface;
determining one or more clinical concepts based on the communication by using natural language processing and an ontological knowledge graph, wherein the ontological knowledge graph defines semantics, constraints, and relationships between a plurality of terms;
querying an electronic health record using the one or more clinical concepts;
receiving, in response to the query, a set of records corresponding to the one or more clinical concepts from the electronic health record;
generating an output based on the set of records by using a generative artificial intelligence (GenAI) model, wherein the output comprises a summary of the set of records, a trend, or a categorization of information of the set of records; and
updating the interface with the output.

10. The system of claim 9, wherein the set of operations further comprises:
generating an inference request for the GenAI model, wherein:
the inference request comprises a prompt and the set of records, or
the inference request comprises the prompt and an identifier for each record of the set of records.

11. The system of claim 9, wherein generating the output based on the set of records by using the GenAI model further includes:
executing an inference request using the GenAI model, wherein the GenAI model is configured to generate an augmented context by combining a prompt with the set of records, and wherein the set of records were not included in training data of the GenAI model; and
generating the output based on the augmented context by using the GenAI model.

12. The system of claim 9, wherein the GenAI model includes a retrieval-augmented generation (RAG) model, a fine-tuned transformer model, or a domain-specific model that is trained on domain-specific data.

13. A computer-program product tangibly embodied in a non-transitory machine-readable storage medium, including instructions configured to cause one or more data processors to perform a set of operations comprising:
accessing a communication that is associated with a subject via an interface;
determining one or more clinical concepts based on the communication by using natural language processing and an ontological knowledge graph, wherein the ontological knowledge graph defines semantics, constraints, and relationships between a plurality of terms;
querying an electronic health record using the one or more clinical concepts;
receiving, in response to the query, a set of records corresponding to the one or more clinical concepts from the electronic health record;
generating an output based on the set of records by using a generative artificial intelligence (GenAI) model, wherein the output comprises a summary of the set of records, a trend, or a categorization of information of the set of records; and
updating the interface with the output.

14. The computer-program product of claim 13, wherein the set of operations further comprises:
generating an inference request for the GenAI model, wherein:
the inference request comprises a prompt and the set of records, or
the inference request comprises the prompt and an identifier for each record of the set of records.

15. The computer-program product of claim 13, wherein generating the output based on the set of records by using the GenAI model further includes:
executing an inference request using the GenAI model, wherein the GenAI model is configured to generate an augmented context by combining a prompt with the set of records, and wherein the set of records were not included in training data of the GenAI model; and
generating the output based on the augmented context by using the GenAI model.

16. The computer-program product of claim 13, wherein the GenAI model includes a retrieval-augmented generation (RAG) model, a fine-tuned transformer model, or a domain-specific model that is trained on domain-specific data.

17. The computer-program product of claim 13, wherein the set of operations further comprises:
receiving one or more selection criteria from a user device, wherein the one or more selection criteria comprises one or more filters corresponding to a time interval, an encounter type, a record type, or an author of a record;
querying the electronic health record using the one or more clinical concepts and the one or more selection criteria;
receiving, in response to the query, a second set of records corresponding to the one or more clinical concepts and the one or more selection criteria; and
generating a second output based on the second set of records by using the GenAI model.

18. The computer-program product of claim 13, wherein the output comprises a problem-oriented summary or a disease-oriented summary based on the set of records.

19. The computer-program product of claim 13, wherein the output comprises the trend of symptoms by using temporal analysis of data from the set of records.

20. The computer-program product of claim 13, wherein the output comprises categorized information of the set of records corresponding to a plurality of sections, wherein the plurality of sections includes symptoms, medications, lab tests, and diagnosis.
